(19) United States
(12) Patent Application Publication
(10) Pub. No.: US 2024/0024628 A1
(43) Pub. Date: Jan. 25, 2024

(54) MEDICAL DEVICE SUPPORT DEVICES

(71) Applicant: ABIOMED, Inc., Danvers, MA (US)

(72) Inventors: Jonathan Barry, Danvers, MA (US); Robert Swierczek, Danvers, MA (US); Anne McLoughlin, Danvers, MA (US); David Johnston, Danvers, MA (US); David Filipiak, Danvers, MA (US); Christopher Korkuch, Danvers, MA (US); Glen Fantuzzi, Danvers, MA (US); Robert Fishman, Danvers, MA (US); Brian Miley, Watertown, MA (US); William Lewis, Watertown, MA (US)

(73) Assignee: ABIOMED, Inc., Danvers, MA (US)

(21) Appl. No.: 18/223,628

(22) Filed: Jul. 19, 2023

Related U.S. Application Data
(60) Provisional application No. 63/390,554, filed on Jul. 19, 2022.
(60) Provisional application No. 63/447,963, filed on Feb. 24, 2023.

(51) Int. Cl. A61M 25/01; A61M 25/02

(52) U.S. Cl. CPC A61M 25/0105; A61M 25/02; A61M 2025/024

(57) ABSTRACT

A positioning device for maintaining positioning of an introducer hub relative to a repositioning sheath or catheter may be provided. The positioning devices may include a first coupler, a second coupler configured to be removably coupled to a portion of a repositioning sheath or catheter that is proximal to the first coupler, and a portion extending between the first coupler and the second coupler, the extension bridge configured to maintain a fixed axial distance between the introducer hub and a proximal end of the repositioning sheath or catheter. A flexible hinge may be incorporated, where a medical device would be configured to pass through the second coupler, the flexible hinge, and the first coupler.

### CROSS-REFERENCE TO RELATED APPLICATIONS

The present application claims priority to U.S. Provisional Patent Application 63/390,554, filed Jul. 19, 2022, and to U.S. Provisional Patent Application 63/447,963, filed Feb. 24, 2023. The entire contents of the above-referenced applications are incorporated herein by reference.

### TECHNICAL FIELD

The present disclosure is drawn to support structures for medical devices that may extend outwardly from a patient's body, such as positioning devices configured to allow medical devices, catheters, and/or sheaths to be positioned at an access site and positioned relative to the patient and/or to a portion of a medical device or support structure, such as a an introducer hub.

### BACKGROUND

In clinical settings, medical devices may be inserted into a patient at an access point, such as via an access device. Following such treatments, the patient may need to have the medical device and/or sheath left in place. For example, a patient may be transported to an intensive care unit with a blood pump in place and a positioning sheath exposed at the access site.

Current practices include, e.g., inserting the end of a repositioning sheath slightly into a hemostatic valve of a hub of an introducer sheath that is inserted through the access site.

### BRIEF SUMMARY

In various aspects, a positioning device for maintaining axial positioning of an introducer hub relative to a repositioning sheath or catheter may be provided. The positioning device may include a first coupler configured to be removably coupled to a proximal end of an introducer hub. The positioning device may include a second coupler configured to be removably coupled to a portion of a repositioning sheath or catheter that is proximal to the first coupler. The positioning device may be configured to maintain a fixed axial distance between the introducer hub and a proximal end of the repositioning sheath or catheter.

In some embodiments, the positioning device may include an extension bridge extending between the first coupler and the second coupler,

In some embodiments, the first coupler may include a first surface containing a threaded portion configured to interact with one or more threads on an exterior surface of the introducer hub to prevent the first coupler from moving axially relative to the introducer hub. In some embodiments, the first coupler may include at least one recess or aperture configured to interact with a tab or extruded portion of an exterior surface of the introducer hub to prevent the first coupler from moving axially relative to the introducer hub. In some embodiments, the first coupler may include at least one fastener configured to interact with a corresponding fastener on an exterior surface of the introducer hub to maintain a position of the introducer hub relative to the first coupler.

In some embodiments, the first coupler may include a rotatable portion and a non-rotatable portion, the rotatable portion located distal from the non-rotatable portion, the rotatable portion configured to interact with threads, tabs, or extruded portions of an exterior surface of the introducer hub. In some embodiments, the first coupler may have an outer diameter greater than an outer diameter of the introducer hub.

In some embodiments, the first coupler may have a constant outer diameter between a first end and a second end. In some embodiments, an outer diameter of a first end of the first coupler may be smaller than an outer diameter of a second end of the first coupler. In some embodiments, an outer diameter of the first coupler may decrease in a direction from a portion in between the first and second ends of the first coupler to the first end of the coupler.

In some embodiments, the positioning device may include an adjustable arm coupled to the first coupler. The adjustable arm may be configured to support the repositioning sheath or catheter as the repositioning sheath or catheter extends proximally from the first coupler.

In some embodiments, the extension bridge may be configured to avoid contacting the repositioning sheath or catheter. In some embodiments, the extension bridge may circumferentially surround the repositioning sheath or catheter. In some embodiments, the extension bridge may partially circumferentially surround the repositioning sheath or catheter.

In some embodiments, the extension bridge may be configured to allow only a portion of a repositioning sheath or catheter to be slidably inserted through the extending section. In some embodiments, the extension bridge may be configured to prevent the repositioning sheath from extending into the introducer hub.

In some embodiments, the extension bridge may have a stiffness greater than a stiffness of the repositioning sheath or catheter.

In some embodiments, the second coupler may be configured to allow only a portion of a repositioning sheath or catheter to be slidably inserted through the coupling section. In some embodiments, the second coupler may include a molded portion that is molded such that an internal surface of the molded portion substantially matches an external surface of the repositioning sheath or catheter. In some embodiments, the molded portion may be configured to encompass at least part of a suture pad. In some embodiments, the molded portion may be configured to encompass at least part of connector at proximal end of the repositioning sheath.

In some embodiments, the positioning device may include a hinge that hingedly connects a first section of the positioning device to a second section of the positioning device, wherein at least a portion of the introducer hub and at least a portion of the repositioning sheath or catheter can be placed at least partially into the first section before the second section hingedly closes. In some embodiments, the positioning device may include a locking mechanism configured to prevent the positioning device from hingedly opening. In some embodiments, the locking mechanism comprises a snap hinge on the first section, the snap hinge comprising a handle and a lock portion configured to slidably couple to a recessed or extruded portion on the second section.

In some embodiments, at least a portion of the introducer hub may be placed at least partially in a portion of the first section forming a portion of the first coupler and at least a portion of the repositioning sheath or catheter is placed in a portion of the first section forming a portion of the second coupler of the first section.

In some embodiments, an inner surface of the first section and/or second section may include an indicator representing a correct positioning of the repositioning sheath.

In some embodiments, each of the first and second sections may include a length having one or more ribs extending into a channel formed between the first and second sections, the channel configured to receive at least a portion of the repositioning sheath and/or catheter.

In some embodiments, the first coupler may include a clamshell and the second coupler may attach to the clamshell via a ball joint that is secured within the clamshell.

In some embodiments, the extension bridge may include a hinged section configured to allow a proximal end of the extension bridge to rotate around the hinged section relative to a distal end of the extension bridge. In some embodiments, the hinged section can be unlocked to allow the hinged section to rotate, or locked to prevent the hinged section from rotating.

In some embodiments, a kit may be provided, the kit may include an introducer hub, a repositioning sheath, and a positioning device as disclosed herein.

In some embodiments, a positioning device may be provided that may be configured for adjustable positioning of a medical device, repositioning sheath, or both, relative to an introducer hub. The positioning device may include a first coupler configured to be removably coupled to a proximal end of an introducer hub. The second coupler may be configured to receive the medical device, repositioning sheath, and/or catheter. The positioning device may include a hinged section configured to allow the adjustable locking portion to rotate around the hinged section relative to the first coupler.

In some embodiments, the second coupler may be configured to allow or prevent the medical device and/or repositioning sheath from being adjusted axially relative to the introducer hub based on a position of an adjustable lock, where the positioning device is configured such that a medical device can be slidably received through the adjustable locking portion, and through the first coupler.

In some embodiments, the hinged section can be unlocked to allow the hinged section to rotate, or locked to prevent the hinged section from rotating.

In some embodiments, a positioning device configured for adjustable positioning of a catheter device, repositioning sheath, or both, relative to an introducer hub may be provided. The positioning device may include a first coupler that may be configured to be removably coupled to a proximal end of an introducer hub. The first coupler may be configured to slidably receive a medical device and allow the medical device to enter into the introducer hub. The positioning device may include a proximal end configured to slidably receive the medical device. The positioning device may include a flexible hinge connecting the first and second couplers, where the flexible hinge may be configured to allow the medical device to be slidably received through the flexible hinge.

In some embodiments, the flexible hinge can be unlocked to allow the flexible hinge to rotate, or locked to prevent the flexible hinge from rotating.

In some embodiments, the flexible hinge may include an outer housing having a distal end, a proximal end, and an inner surface defining a first volume of space extending therethrough. In some embodiments, the flexible hinge may include a joint (which may be a ball joint) having a distal end, a proximal end, and an inner surface defining a second volume of space therethrough. The distal end of the joint may be positioned at least partially within the first volume of space. The joint may be configured to rotate relative to the outer housing around a first axis, the first axis being orthogonal to a path from the first end of the outer housing to the second end of the outer housing. The second volume of space may be configured to allow a medical device to be slidably received and directed from the proximal end of the joint to the distal end of the outer housing.

In various aspects, a positioning device may be provided. The positioning kit may include a first coupler. The first coupler may have a housing. The first coupled may have a means for locking the first coupler to an introducer sheath. The first coupler may include an alignment tab. The first coupler may include one or more indicia of a locking status of the first coupler.

The positioning device may include a second coupler. The second coupler may be connected to the first coupler. The second coupler may be configured to receive a tubular member. The first coupler may be pivotably attached to the second coupler.

In various aspects, a kit may be provided. The kit may include a medical device, a positioning device as disclosed herein, and optionally a repositioning sheath.

In various aspects, a method for adjusting positioning of a medical device, repositioning sheath, or both relative to an introducer hub may be provided. The method may include adjusting the positioning of a medical device, repositioning sheath, or both. The adjusting may include: rotating a proximal end of the positioning device relative to the distal end of the positioning device; moving the medical device, repositioning sheath, or both in an axial direction; or a combination thereof. The method may include locking the locking mechanism on the positioning device to prevent the medical device, repositioning sheath, or both from moving axially relative to the locking mechanism.

In some embodiments, the method may include unlocking a locking mechanism on the positioning device. In some embodiments, the method may include attaching the first coupler to the hub before adjusting. In some embodiments, the method may include attaching the second coupler to the repo sheath and/or catheter after adjusting.

In some embodiments, locking the locking mechanism on the positioning device may include: locking a first locking mechanism to prevent the medical device, repositioning sheath, or both from moving axially relative to the locking mechanism; and locking a second locking mechanism to prevent the proximal end of the positioning device from rotating relative to the distal end of the positioning device.

In some embodiments, the flexible hinge may include an outer housing having a distal end, a proximal end, and an inner surface defining a first volume of space extending therethrough. In some embodiments, the flexible hinge may include a joint having a distal end, a proximal end, and an inner surface defining a second volume of space therethrough. The distal end of the joint may be positioned at least partially within the first volume of space. The joint may be configured to rotate relative to the outer housing around a first axis, the first axis being orthogonal to a path from the first end of the outer housing to the second end of the outer housing. The second volume of space may be configured to allow a medical device to be slidably received and directed from the proximal end of the joint to the distal end of the outer housing.

In various aspects, a system may be provided. The system may include a flexible hinge operably coupled to an introducer hub, and a medical device configured to pass through the flexible joint and into the introducer hub. The flexible hinge may include an outer housing having a distal end, a proximal end, and an inner surface defining a first volume of space extending therethrough. In some embodiments, the flexible hinge may include a joint having a distal end, a proximal end, and an inner surface defining a second volume of space therethrough. The distal end of the joint may be positioned at least partially within the first volume of space. The joint may be configured to rotate relative to the outer housing around a first axis, the first axis being orthogonal to a path from the first end of the outer housing to the second end of the outer housing. The second volume of space may be configured to allow a medical device to be slidably received and directed from the proximal end of the joint to the distal end of the outer housing.

In various aspects, a method for coupling and/or decoupling a coupler and a hub may be provided (e.g., for preventing a medical device, repositioning sheath, or both from moving axially relative to the hub, or allowing the medical device and/or repositioning sheath to be moved). The method may include aligning and engaging a first coupler to a hub as disclosed herein. In some embodiments, a medical device and/or repositioning sheath may be introduced before the first coupler is aligned and engaged. In some embodiments, a medical device and/or repositioning sheath may be introduced after the first coupler is aligned and engaged. The method may include moving at least a portion of the first coupler or the hub from a first position to a second position.

In some embodiments, this may be from an unlocked position to a locked position. In some embodiments, this may be from a locked position to an unlocked position.

### BRIEF DESCRIPTION OF DRAWINGS

FIG. 1A is an illustration of an embodiments of a positioning device.

FIGS. 1B and 1C are partial cut-away illustrations of first couplers of positioning devices.

FIGS. 1D and 1E are illustrations of embodiments of a positioning device.

FIGS. 1F and 1G are illustrations of a positioning device aligned and locked onto a hub, respectively.

FIG. 1H illustrate a positioning device and an introducer hub according to one embodiment.

FIGS. 1I-1L illustrate positioning devices according to other embodiments of the present disclosure.

FIG. 1M illustrates a portion of a positioning device according to one embodiment.

FIG. 2A is an illustration of an embodiment of a positioning device with a clamshell design in an open configuration.

FIG. 2B is an illustration of an embodiment of a positioning device with a clamshell design in a closed configuration.

FIG. 2C is an illustration of an embodiment of a positioning device with a clamshell design in an open configuration.

FIG. 2D is an illustration of the positioning device of FIG. 2C in a closed configuration.

FIG. 3A is an illustration of an embodiment of a positioning device with a flexible hinge design.

FIG. 3B is an illustration of a cross-sectional view focusing on the hinge portion of an embodiment with a flexible hinge design.

FIG. 3C is an illustration of an embodiment of the positioning device of FIG. 3B, where the hinge is adjusted downward towards the patient's skin.

FIG. 3D is an illustration of a positioning device according to one embodiment.

FIG. 3E is an illustration of the positioning device of FIG. 3D in a locked position.

FIG. 3F is an illustration of a positioning device according to another embodiment.

FIG. 3G is an illustration of a positioning device according to another embodiment.

FIG. 3H is an illustration of a positioning device according to another embodiment.

FIG. 3I is an illustration of the positioning device of FIG. 3D in a locked position.

FIG. 4 is a flowchart of a method.

FIGS. 5A and 5B are illustrations of an embodiment with a graft.

### DETAILED DESCRIPTION

As is known, medical devices may be inserted into a patient at an access point, such as via an access device, to perform a medical procedure or treatment. Following such procedures or treatments, the patient may need to have the medical device and/or sheath left in place. For example, a patient may be transported to an intensive care unit with a blood pump in place and a positioning sheath exposed at the access site.

In some embodiments, an end of a repositioning sheath may be inserted slightly into a hemostatic valve of a hub of an introducer sheath that is inserted through the access site. To avoid issues of contamination, bleeding, and/or blockage of the side ports, the inventors have recognized the benefits of a devices that may allow for proper positioning and securement of the medical device (e.g., pump), repositioning sheath, and/or an anti-contamination barrier at an access site. For example, the inventors have recognized the benefit of a device that may minimize and/or prevent movement relative to an introducer hub and/or to the patient. IN some examples, the device may be configured to minimize and/or prevent axial and/or radial movement relative to the introducer hub and/or patient. In some embodiments, a positioning device for maintaining axial positioning of an introducer hub relative to a repositioning sheath or catheter may be provided.

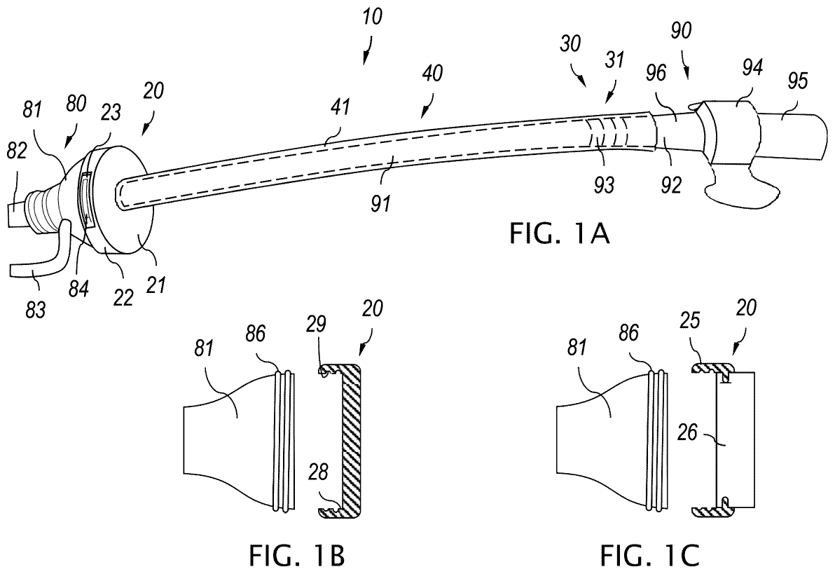

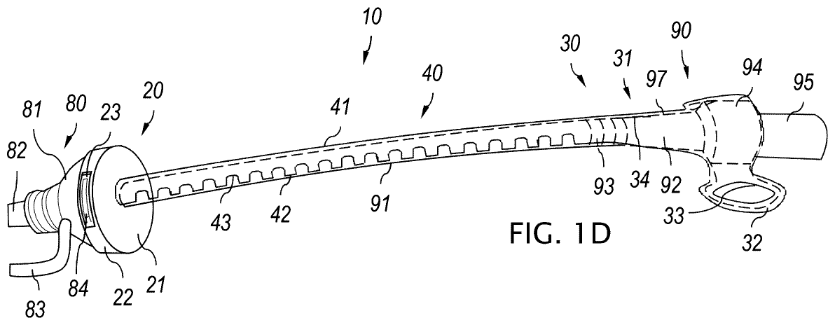

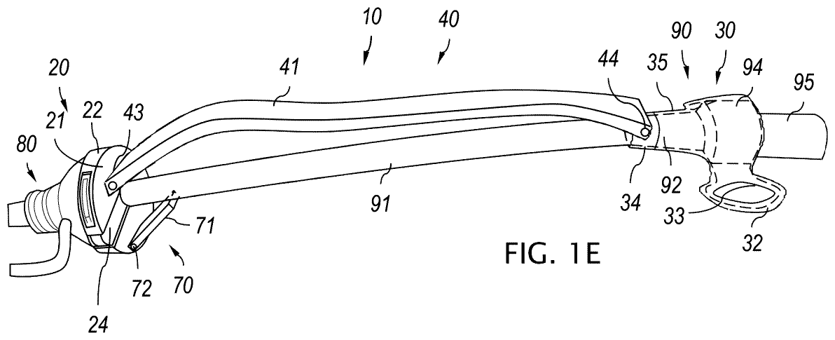

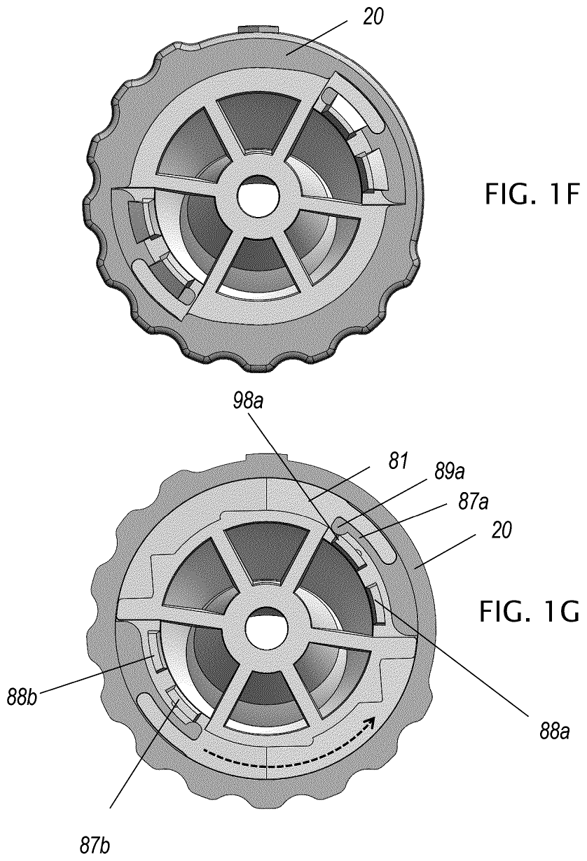

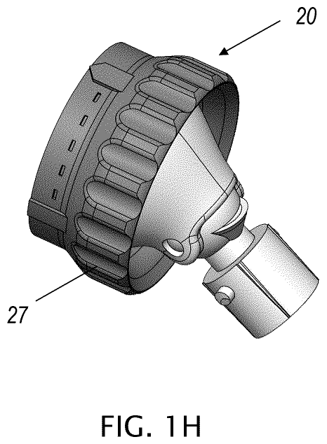

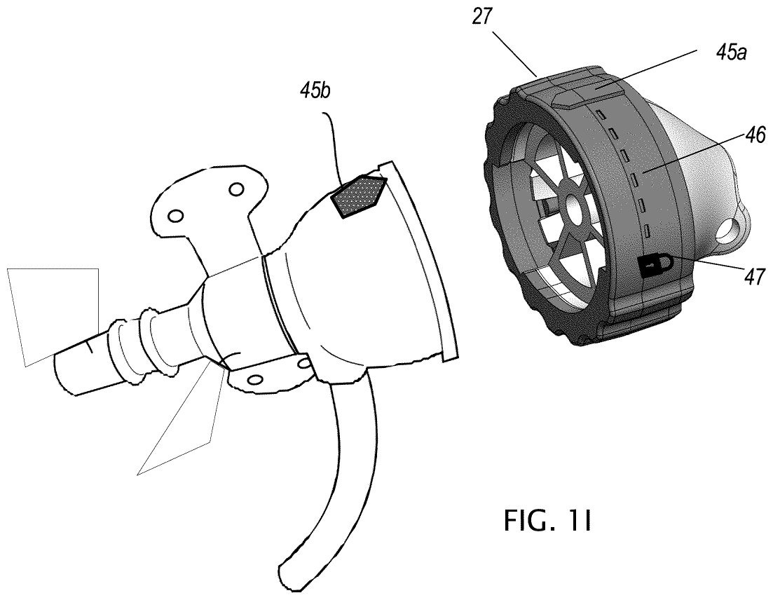

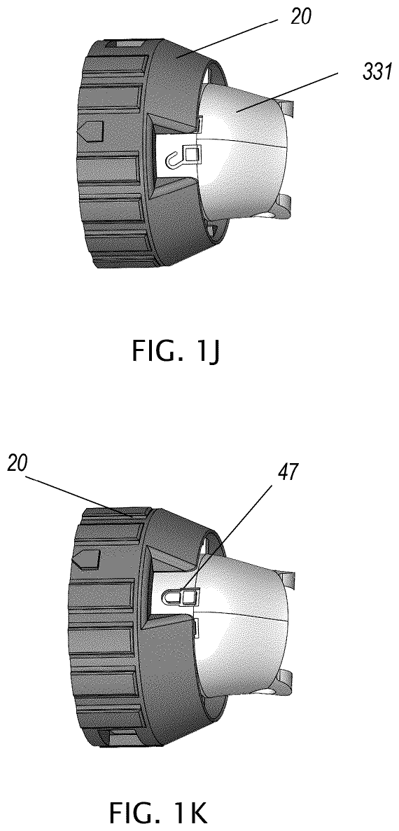

Turning now to the figures, FIG. 1A shows an embodiment of a positioning device according to embodiments of the present disclosure. In some embodiments, as shown in this figure, a positioning device 10 may include a first coupler 20 (e.g., a distal coupler or distal end coupler) configured to be removably coupled to a hub body 81 of an access device, such as introducer 80, for example to the proximal end of the hub body. In some instances, the introducer may have a sheath 82 coupled to a distal end of the hub body 81 and may have a side port 83 extending away from a side surface of the hub body 81.

In some embodiments, the positioning device may include a second coupler 30 configured to be removably coupled to a portion of a repositioning sheath 90 or catheter that is proximal to the first coupler.

The positioning device may include a bridge extension 40 that extends between the first coupler 20, (e.g., the distal end coupler) and the second coupler 30. The bridge extension may be configured to maintain a fixed distance (e.g., axial distance) between the proximal end of the introducer hub 81 and a proximal end of the repositioning sheath 90 or catheter.

In some embodiments, the first and second couplers and the bridge extension may be permanently attached, such as integrally formed, with one another. In other embodiments, one or both of the first and second couplers may be removably attachable to the bridge extension. In this regard, the positioning device may include a modular system, which may be configurable by a clinician.

Any appropriate method for connecting the first coupler 20 to the introducer hub, and for maintaining a desired position of the first coupler relative to the introducer hub are envisioned.

For example, referring briefly to FIG. 1B, in some embodiments, the first coupler may be configured to threadably engage with the hub. For example, the first coupler 20 may include a chamber having a first surface 28 containing a threaded portion (e.g., including recesses 29) configured to interact with one or more threads 86 on an exterior surface of the introducer hub 81 to prevent the first coupler from moving (e.g., axially) relative to the introducer hub. As will be appreciated, other suitable engagement methods may be used to attach the coupler to the hub. For example, in some embodiments, the coupler can be press-fit or snap fit to the hub.

As will be appreciated, in some embodiments, engagement between the coupler and the hub may be sufficient to lock the coupler to the hub and maintain the position of the coupler relative to the hub. In other embodiments, as described herein, the coupler may include one or more locking features to lock the prevent movement between coupler and the hub. For example, in some embodiments, the first coupler also may include at least one recess or aperture 23 configured to interact with a tab or extruded portion 84 of an exterior surface of the introducer hub 81 such as to prevent the first coupler from moving (e.g., axially) relative to the introducer hub. As will be appreciated, the hub also may include an aperture which is configured to engage with one or more tabs or extruded portions on the first coupler. In this regard, via the threaded engagement and the tab(s), the coupler may be locked relative to the hub.

In other embodiments, as shown by way of example in FIGS. 1F and 1G, the proximal face of the coupler may include one or more fasteners that interact with corresponding fasteners on the distal face of the hub to lock the distal hub to the coupler as the coupler is rotated into a locked position (see the arrow in FIG. 1G). For example, as shown in these views, the coupler may include first and second arms 87a, 87b that are slidably received within first and second fasteners 88a, 88b, respectively (e.g., locking bumps) on the hub. In such embodiments, a contacting portion 89a of the first and second arms (e.g., a distal portion) may be configured to contact a corresponding contacting portion 98a of the corresponding fastener (e.g., a contacting edge) to lock the position of the arms relative to the faster and, in turn, the position of the hub relative to the coupler. As will be appreciated, the corresponding contacting portions may have any suitable shape and size for engaging with one another.

Although shown with two arms and four corresponding fasteners, it will be appreciated that the hub and first coupler may have any suitable numbers of corresponding fasteners. Further, although the hub is shown with fasteners for slidably receiving arms on the coupler, in other embodiments, the coupler may include one or more fasteners for receiving arms on the hub. In still other embodiments, other cantilevered arrangements may be used to lock the coupler relative the hub. As will be appreciated, in all such embodiments, the first coupler may be unlocked from the hub, such as after completion of a treatment or procedure.

Referring briefly to FIG. 1C, in some embodiments, the first coupler may include a rotatable portion 25 and a non-rotatable portion 26, the rotatable portion located distal from the non-rotatable portion. In some embodiments, the rotatable portion 25 may be configured to interact with threads, tabs and/or extruded portions of an exterior surface of the introducer hub to prevent the first coupler from moving (e.g., axially) relative to the introducer hub (in FIG. 1C, the rotatable portion includes recesses intended to interact with threads located on an outer surface of the hub). As will be appreciated, the shape, size and/or pitch of the threads on the first coupler may correspond to the shape, size, and/or pitch of the threads on the hub such that the first coupler may be coupled to the hub.

In some embodiment, as shown in FIGS. 1H and 1I, the first coupler 20 may include a gripping region 27 to assist a clinician in moving the coupler relative to the hub. As will be appreciated, the gripping region may extend around an entire circumference of the coupler (see FIG. 1H) or around only a portion of the circumference of the coupler (see FIG. 1I). In some embodiments, the gripping region may include a plurality of axially extending protrusions (e.g., ribs) and/or depressions (e.g., channels), which may alternate around the circumference of the coupler. As will be appreciated, each of the protrusions may be the same size, although they also may vary from protrusion to protrusion. In a similar fashion, each of the depressions may be the same size or they may vary from depression to depression.

In some embodiments, as shown in FIG. 1I, for example, the coupler may include an alignment tab 45a, which may be used by a clinician to properly align the coupler on the hub. For example, in some embodiments, as shown in FIG. 1I, a user may align the alignment tab 45a of the coupler with a corresponding alignment tab 45b on the hub and may then engage the coupler with the hub. In some embodiments, once aligned and engaged, the coupler may be locked to the hub such that position of the coupler relative to the hub may be maintained. In other embodiments, when the coupler is aligned and engaged with the hub, the coupler may not be locked to the hub. Instead, the coupler may have a first or “home” position when aligned and engaged and a second “locked” position. In some embodiments, the clinician may use the coupler from the first position to the second position to lock the coupler relative to the hub.

In some embodiments, the coupler may include indicia (e.g., visual and/or audible) to indicate when the coupler moving between the first and second position 46 and also to indicate when the coupler is in the locked position 47. As will be appreciated, other suitable indicia may be utilized in other embodiments.

In some embodiments, the first coupler may have an outer diameter greater than an outer diameter of the introducer hub. In some embodiments, the first coupler may have an outer diameter greater than an outer diameter of the introducer hub. For example, in FIG. 1B, the threads on the hub are shown on an outward-facing surface (e.g., extending away from a central axis), and the recesses on the first coupler are on an inward-facing surface (e.g., the recesses are on a surface facing a central axis). However, one of skill in the art will understand that the threads could be on an inward-facing surface, where the recesses to receive the threads would be on an outward-facing surface of the first coupler. Similarly, it will be understood that the relevant components involved in coupling the hub to the first coupler (e.g., threads, apertures, etc.) may be on the hub or on the first coupler. For example, if the threads are on the first coupler, the recesses would be on the hub.

In some embodiments, the first coupler may have a substantially cylindrical outer wall (e.g., with a constant diameter between a first end and a second end). In other embodiments, as shown in FIGS. 1J and 1K, a portion of the coupler may have a wall that tapers. For example, as shown in these views, an outer diameter of the coupler may decrease in a direction towards a first end, e.g., in a direction away from the hub. In some embodiments, the outer diameter may decrease from position in between the first and second ends (e.g., mid-way between the first and second ends) and towards the second end (e.g., in a direction away from the side of the coupler that engages with the hub. In some embodiments, the coupler may include one or more cutouts or windows for viewing visual indicia of the locking status of the coupler relative to the hub (see e.g., the lock icon 47 in FIG. 1K).

In some embodiments, as also shown in FIGS. 1J and 1K, the coupler may include a housing 331, which as described herein, may act as a socket and allow the clinician flexibility in adjusting the position of the hub and/or medical device relative to the patient.

As will be appreciated, the coupler may be attached to the housing in any suitable manner. For example, in some embodiments, as shown in FIG. 1L, the coupler may include first and second portions 20a, 20b, with a portion of the housing being sandwiched therebetween. For example, the first and second coupler portions may be threaded together and around the housing to attach the coupler to the housing. The first and second coupler portions also may be snapped together via one or more fasteners. As will be appreciated, the coupler may thereafter be attached to the hub (e.g., via a threaded engagement and/or fasteners.

Although shown as having two coupler portions, it will be appreciated that the coupler may have more than two coupler portions. Also, although the coupler portions are shown as each defining a circumference of the coupler and having different lengths that attach together to form the complete length of the coupler, in other embodiments, each coupler portion may form only a portion of the circumference of the coupler (while having the same length), with the first and second coupler portions attaching together to form the complete circumference of the coupler (see, e.g., FIG. 1M). As will be appreciated, in addition to snapping to engage, the coupler may be pressed to release from the hub.

Although shown as being attachable to the housing, it will be appreciated that the coupler also may be integrally formed with the coupler.

Referring again to FIG. 1A, in some embodiments, the bridge extension 40 may circumferentially surround at least a portion of the repositioning sheath 90 or catheter. In such embodiments, the repositioning sheath or catheter may include a flexible tubular member 91 extending proximally away from a coupling 92. The coupling may have one or more fasteners 93 that are configured to interact with the second coupler 30, for example, to increase friction, provide slots or ridges with which part of the second coupler may interact in order to prevent (e.g., axial) movement of the repositioning sheath or catheter relative to the second coupler 30. In some embodiments, a suture hub 94 may be present on the repositioning sheath or catheter, and may be coupled (e.g., rotatably coupled) to the coupling 92. Additional components 95 of the repositioning sheath or catheter, including other fittings or tubular members, may extend proximally away from the positioning device.

In some embodiments, the bridge extension 40 may circumferentially surround the portion of the flexible tubular member 91 that is between the first coupler 20 and the coupling 92.

Referring to FIG. 1D, in some embodiments, the bridge extension may partially circumferentially surround the repositioning sheath or catheter. As seen in FIG. 1D, in some embodiments, there may be a pattern of recesses or apertures 43 axially positioned along the extension bridge. In some embodiments, the extended portion may include sidewalls that extend only partially around the repositioning sheath or catheter. For example, in some embodiments, at a first point in an axial direction, one edge 42 of the extended portion may be circumferentially separated from a second edge (not shown) by a distance greater than 0. In some embodiments, the circumferential distance is less than half of the total circumference of the repositioning sheath or catheter.

Referring to FIG. 1E, in some embodiments, the bridge extension 40 may be configured to avoid contacting the repositioning sheath or catheter. In some embodiments, the bridge extension may be configured to be not coaxial with the repositioning sheath or catheter. In some embodiments, the bridge extension is not parallel with the repositioning sheath or catheter. For example, in some embodiments, wall(s) 41 defining the shape of the bridge extension 40 may be configured to form a handle, and/or have an ergonomic shape. In some embodiments, the bridge extension may be configured such that at least one wall 41 lies at least partially against a patient's skin.

In some embodiments, the bridge extension is coupled directly to the first coupler. In some embodiments, the bridge extension may be coupled to the first coupler 20 indirectly. For example, in some embodiments, a hinge 43 may connect the bridge extension to the first coupler (see FIG. 1E). In such embodiments, a clinician may be able to adjust a position of the bridge extension relative to the first coupler.

In some embodiments, the bridge extension may be coupled directly to the second coupler 30. In some embodiments, the bridge extension is coupled to the second coupler indirectly. For example, in some embodiments, a hinge 44 also may connect the bridge extension to the second coupler 30. As with the above, in such embodiments, this may allow the clinician to adjust a position of the bridge extension relative to the second coupler.

In some embodiments, the second coupler 30 may include a rotatable portion 34 and a non-rotatable portion 35. The rotatable portion may be configured to rotate around a central axis of the non-rotatable portion. In some embodiments, hinge 44 may connect to the rotatable portion.

Referring to FIG. 1E, in some embodiments, the positioning device 10 may include an adjustable arm 70 coupled to the first coupler 20. The adjustable arm 70 may be configured to support the repositioning sheath 90 and/or catheter (and the flexible tubular member 91 in particular) as the repositioning sheath or catheter extends proximally from the first coupler.

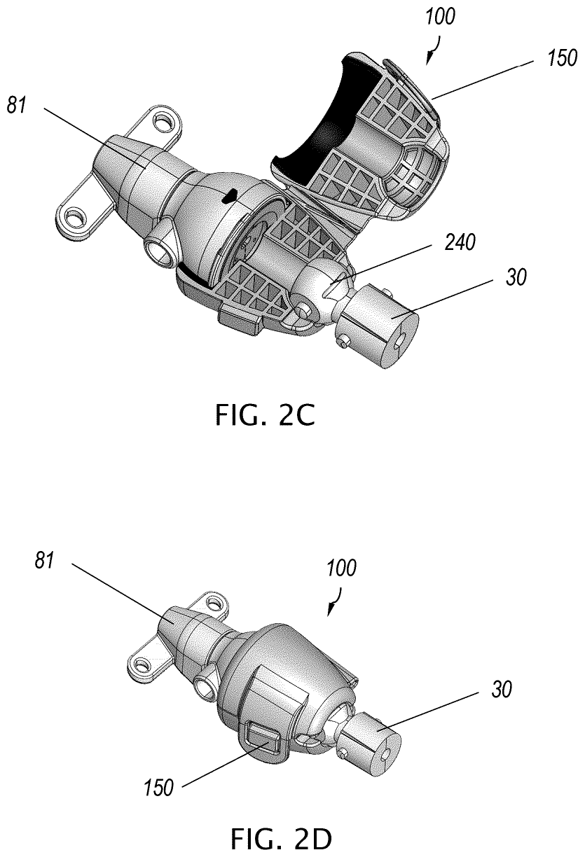

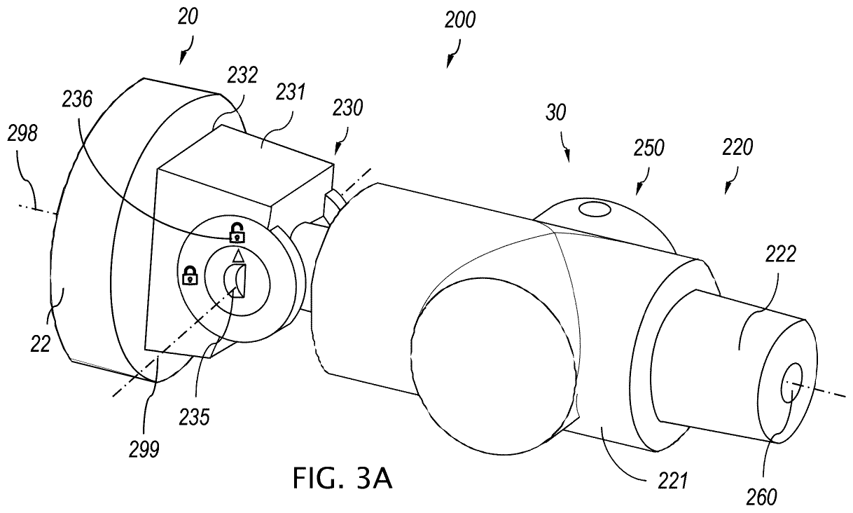

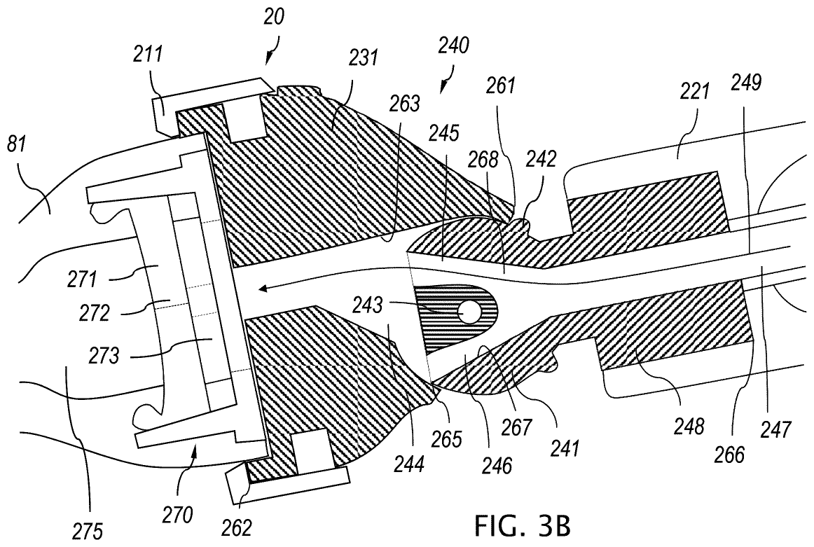

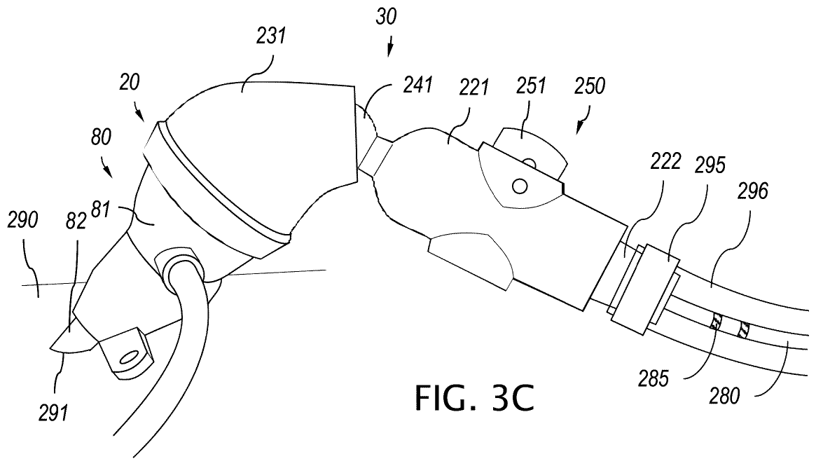

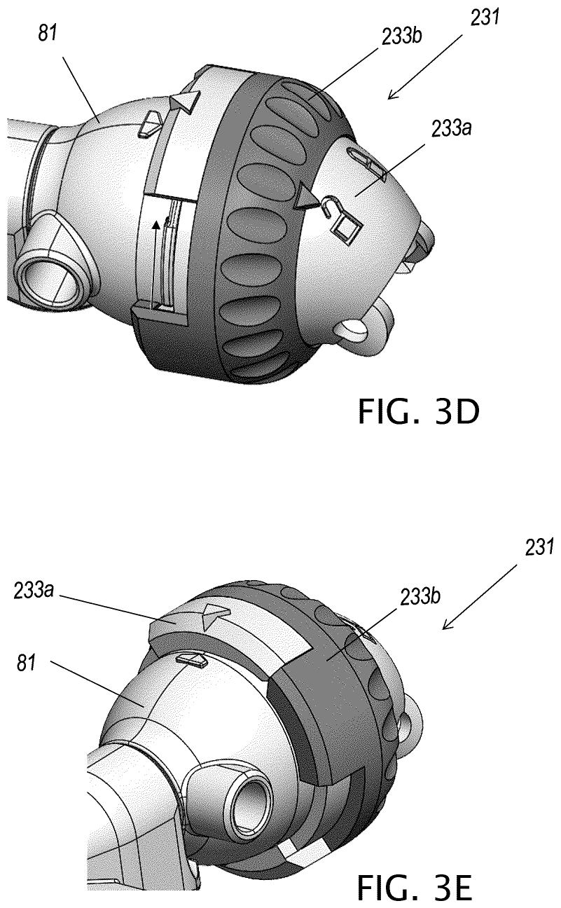

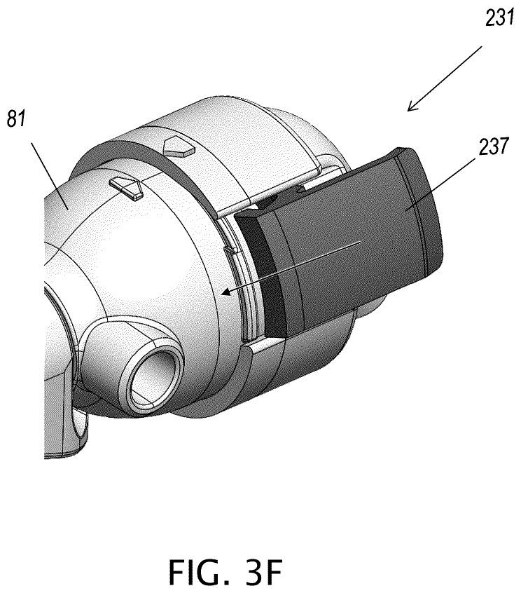

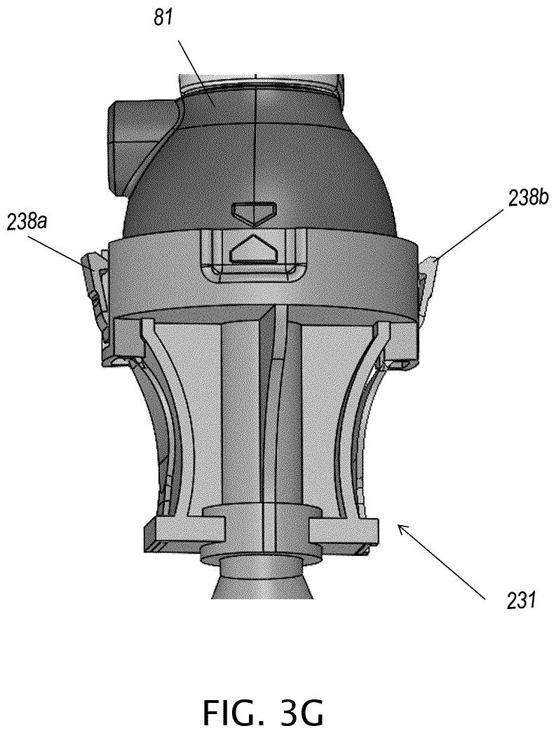

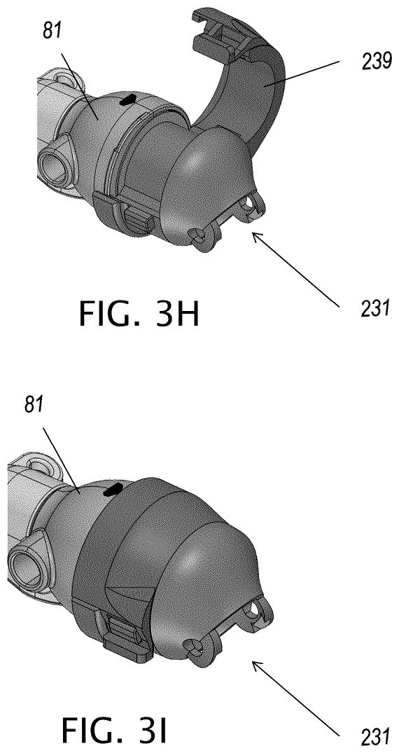

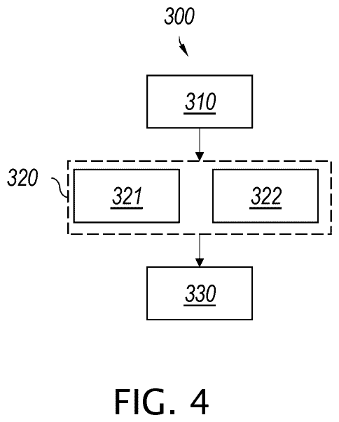

In some embodiments, the first coupler 20 may include a proximal surface 21 and side surface 22 that define a slot 24 (or aperture, etc.) extending through a portion of the first coupler 20. The slot 24 may be configured to allow the device to be attached after a repositioning sheath or catheter are in place. For example, in such embodiments, the flexible tubular member 91 may be slidable through the slot 24 as the first coupler 20 is coupled to the hub. As described herein, and as will be appreciated in view of FIGS. 1H-1L and FIGS. 3A and 3B, the coupler also may be coupled to a housing and/or joint through which the tubular member may be slidable.

In some embodiments, the bridge extension may have a stiffness greater than a stiffness of the repositioning sheath and/or catheter. In some embodiments, the bridge extension may have a stiffness greater than a predetermined stiffness sufficient to prevent the extension bridge from deflecting from the weight of the repositioning sheath or catheter when in use. In such embodiments, the bridge extension may still be formed of a flexible material.

Referring again to FIG. 1A, in some embodiments, the second coupler 30 may be configured to allow only a portion of a repositioning sheath 90 or catheter to be slidably inserted through the coupling section. For example, in some embodiments, the flexible tubular member and a first portion 93 of the coupling may be configured to be slidably received by the second coupler. A second portion 96 of the coupling may be unable to be inserted further through the coupling section.

In some embodiments, the second coupler may include a molded portion 31 configured to encompass at least a portion of the coupling.

Referring to FIG. 1D, in some embodiments, the second coupler 30 may include a molded portion 31 that is molded such that an internal surface 34 of the molded portion substantially matches a portion of an external surface 97 of the repositioning sheath or catheter.

In some embodiments, the molded portion of the second coupler may include a section 32 configured to encompass at least part of the suture pad 94. In some embodiments, the section 32 may define one or more apertures 33 to allow direct access to the suture pad.

In some embodiments, the molded portion of the second coupler 30 may be configured to encompass at least part of an additional component 95 (e.g., a connector, tube, etc.) at a proximal end of the repositioning sheath.

In some embodiments, the second coupler is configured to be placed over or around a portion of the repositioning sheath or catheter, where that portion of the sheath or catheter is not slidably inserted into the second coupler. For example, in some embodiments, a molded portion of the second coupler may be pressed over the coupling of the repositioning sheath or catheter until that portion of the coupling is secured in the second coupler. In that regard, embodiments, the second coupler may be configured to be snapped onto at least a portion of the repositioning sheath, with the second coupler having a press or snap fit engagement with the repositioning sheath and/or catheter.

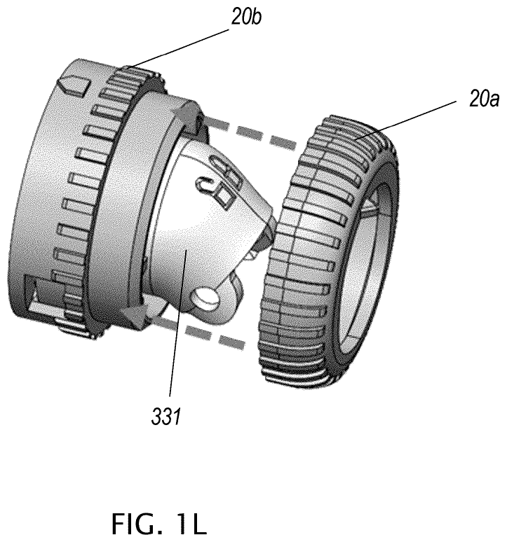

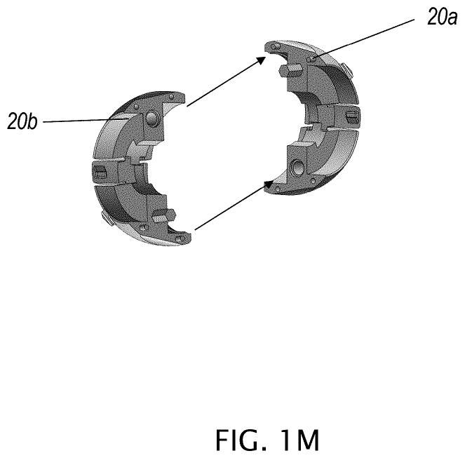

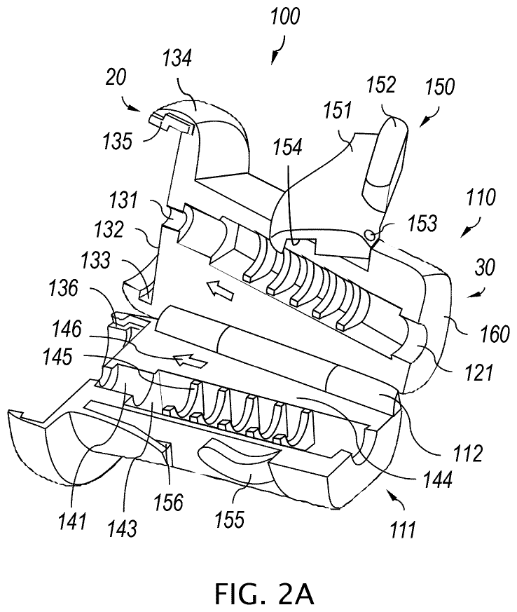

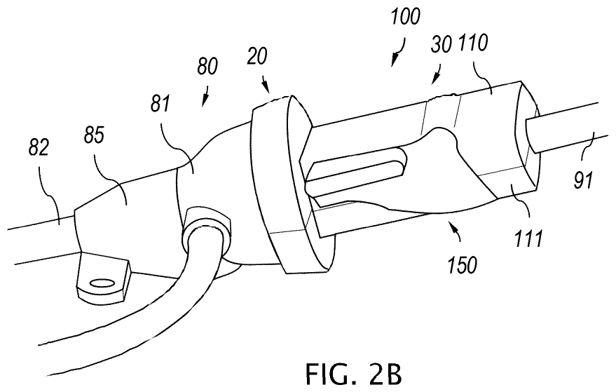

FIGS. 2A and 2B illustrate another embodiment of a positioning device according to the present disclosure. As shown in these views, the positioning device may include a clamshell design and may be configured to engage with the hub and to hold a tubular member relative to the positioning device and to the hub. In this regard, the clamshell may be configured to function as both the first and second couplers 20, 30. As shown in these views, the positioning device 100 will generally be separated into two sections 110, 111 connected via a hinge 112.

As seen in FIGS. 2A and 2B, some embodiments may just include the first coupler and second coupler, and do not include an extension bridge.

As seen in FIG. 2A, the first coupler (e.g., distal end coupler) includes a first surface 132 that may define an opening 131 extending therethrough when in use (e.g., when the two sections 110, 111 are configured in a closed position so the two inner surfaces 144 of the device are in contact with each other). The first coupler 20 may have sidewalls 134 that define one or more recesses 133 for interacting with threads, tabs, etc., on an exterior surface of an introducer hub 81 (see, e.g., the first coupler in FIGS. 1A-1E). As shown in FIG. 2B, the introducer 80 may include a rotatably attached suture hub portion 85.

In some embodiments, as shown in FIG. 2A, the first coupler may have raised portion 135 on one of the two sections 110, 111 configured to fit into recessed portions 136 on the other of the two sections.

The second coupler 30 may include a length of the positioning device proximal to the first coupler. For example, the second coupler and the first coupler may be adjacent to one another. In some embodiments, the second coupler 30 may include a proximal surface 160 that defines a proximal opening 121 extending therethrough when in use. In some embodiments, the proximal opening 121 may have a larger diameter than the distal opening 131. As will be appreciated, a portion of the repositioning sheath may be configured to extend outwardly through the proximal opening 121.

In some embodiments, a length of each section 110, 111 of the positioning device of the second coupler 30 may include an opening extending therethrough when in use to receive a portion of the repositioning hub (or another tubular structure). In some embodiments, the length may include one or more ribs 145, each rib separated axially from other ribs, and each rib molded to allow a repositioning sheath or catheter to be slidably received. In some embodiments, the ribs may maintain the position of a sheath or catheter relative to the positioning device 100.

In some embodiments, the second coupler 30 may be configured to allow only a portion of a repositioning sheath or catheter to be slidably inserted through the second coupler. For example, in some embodiment, the second coupler may include an opening 141 with a diameter that narrows in the distal direction, the diameter of the opening being narrower than the proximal opening and also the outer diameter of the repositioning sheath and/or catheter such that the opening serves as a stopper for the repositioning sheath. In that regard, the most distal end of the repositioning sheath cannot extend beyond the distal end of the opening 141. In some embodiments, the most distal end of the repositioning sheath cannot extend beyond a proximal surface 143 that defines the proximal entrance to opening 141. In this regard, the proximal surface of the opening may serve as a stopper ledge. In some embodiments, the positioning device (e.g., the extension bridge, or the extension bridge in combination with the first coupler) may be configured to prevent the repositioning sheath from extending into the introducer hub.

As will be appreciated, although shown as having the opening 141 with the smaller diameter to serve as the stopper for the repositioning sheath and/or ledge, it will be appreciated that the second coupler may simply have a stopper ledge that extends into the channel (similar to that of the ribs albeit to a positioning closer to the central axis of the channel) to stop the repositioning sheath and/or catheter from extending pas the ledge.

In some embodiments, the positioning device may include a hinge 112 that hingedly connects a first section 110 of the positioning device to a second section 111 of the positioning device. In this way, when in an open configuration (such as that seen in FIG. 2A), at least a portion of the introducer hub and at least a portion of the repositioning sheath or catheter can be placed at least partially into the first section 110 before the second section hingedly closes to be in the closed configuration (such as that seen in FIG. 2B).

In some embodiments, on an inner surface 144 of the first section 110 and/or the second section 111, the positioning device may include an indicator 146 representing a correct positioning of the repositioning sheath. This indicator may include one or more icons, lines, or other human-detectable means of indicating where and/or how a repositioning sheath or catheter should be placed in the device. In some embodiments, the positioning device may include a window through which a clinician may view the repositioning sheath and/or catheter in the positioning device to determine appropriate position.

In some embodiments, the positioning device may include a lock 150 configured to prevent the positioning device from hingedly opening after it is placed in a closed configuration. In some embodiments, the lock may include a snap hinge on one of the clamshell sections, such as on the first section 110. In some embodiments, the snap hinge may include a handle 152 and a lock portion 154 configured to slidably couple to a recessed 156 and/or extruded 155 portion on the second section. The snap hinge may rotate around a hinge pin 153.

As seen in FIG. 2A, the lock is in a first, open position, allowing the positioning device to be freely open or closed. In FIG. 2B, the lock has been moved (here, rotated) to be in a second, closed position, thereby locking the positioning device in the closed configuration, until the lock is moved back to the first, open position.

In some embodiments, hinge pin 153 of the lock extends in a direction perpendicular to the hinge pin of the hinge 112 connecting the first and second sections 110, 111 of the positioning device. In some embodiments, the hinge pin 153 of the lock extends perpendicular to that of the hinge pin 112 connecting the first and second sections of the positioning device. In this regard, the lock moves in a different direction than the direction in which the first and second sections open and close relative to one another.

According to other embodiments, to provide even further control over the positioning and movement of a repositioning sheath or catheter, certain additional elements may be added to the positioning device. For example, in some embodiments, a positioning device may be configured for adjustable positioning of a medical device, repositioning sheath, or both, relative to an introducer hub may be provided. In such embodiments, the first and second couplers may be moveable relative to one another such that the position of the repositioning sheath and/or catheter may be adjustable relative to the hub.

FIGS. 2C and 2D show another exemplary positioning device. Similar to FIGS. 2A and 2B, the positioning device 100 in FIGS. 2C and 3D also includes a clamshell design that can be hingedly opened and closed and locked (see lock 150). In FIGS. 2C and 2D, however, instead of being directly coupled to a flexible tubular member, the clamshell may be attached to the hub and to a ball joint 241. In this regard the positioning device may again act similar to the first couplers described herein and may be configured to maintain a position of the hub relative to the medical device, and the ball joint may be attached to the second coupler for receiving the tubular member. The first coupler may be pivotably attached to the second coupler. As will be appreciated in view of FIGS. 2C and 2D, the ball joint may allow a position of the hub relative to the patient to be adjusted, as the ball joint may allow for rotation of the tubular member that may be slidable therethrough and through the hemostatic valve of the hub.

Referring to FIG. 3A, in some embodiments, the positioning device may include a hinge 230 connecting the first coupler 20 and second coupler 30. In such embodiments, the hinge 230 (sometimes referred to as a “flexible hinge”) may be configured to allow the second coupler to rotate around the hinge (see hinge axis 299 in FIG. 3A) and relative to the first coupler. In such embodiments, the hinge axis may extend perpendicular to a central, longitudinal axis 298 of the positioning device.

In some embodiments, the hinge may include a lock 235 that can be moved to an unlocked position to allow the hinged section to rotate, or moved to a locked position to prevent the hinged section from rotating. The lock may include visual indicia, e.g., icons 236 or other visual representations, indicative of whether the hinged section is locked or unlocked.

As with the above, the first coupler 20 of FIG. 3A may be configured to be removably coupled to a proximal end of an introducer hub. The first coupler also may be configured to slidably receive a medical device and allow the medical device to enter into the introducer hub.

The second coupler 30 of the positioning device may include a proximal end 220 configured to slidably receive the medical device. The proximal end may include a first section 221 and a second section 222, where the second section has a smaller outer diameter than the first section. The proximal end of the second section may define an opening 260 that is configured to slidably receive a medical device.

In some embodiments, the second coupler may include the proximal end 220 and an adjustable lock 250. The adjustable lock may be configured to allow or prevent the medical device and/or repositioning sheath from being adjusted axially relative to the second coupler 30 and to the introducer hub based on a position of the adjustable lock.

Referring to FIG. 3B, a close-up of a hinge having a ball joint can be seen. In some embodiments, the hinge 240 may include an outer housing 231 having a distal end 262, a proximal end 261, and an inner surface 263 defining a first volume of space 244 extending therethrough. The outer housing may also define a part of the first coupler 20 (see also FIG. 1H-1M). As seen in FIG. 3B, the first coupler may include a ring 211 that may be rotatable coupled to the outer housing 231 of the hinge. In some embodiments, a portion of ring 211 may be configured to interact with an exterior surface of the introducer hub 81. For example, the ring 211 may be threaded onto the hub in some embodiments. As will be appreciated, the ring also may be fixedly attached to the outer housing 231 of the ring.

As shown in FIG. 3B, the flexible hinge may include a ball 241 having a distal end 265, a proximal end 266, and an inner surface 267 defining a second volume of space 268 therethrough. In some embodiments, the distal end 265 of the ball 241 may be positioned at least partially within the first volume of space 244 of the outer housing. In some embodiments, the ball 241 may include one or more raised portion 242 configured to prevent the ball from rotating beyond a predefined amount. As will be appreciated, the shape and size of the ball may correspond to the shape and size of the first volume of space 244.

The proximal end 266 of the ball (e.g., the ball 241) may be coupled to the first section 221 of the second coupler 220 of the device.

In some embodiments, the ball joint may allow rotation of the second coupler in any direction relative to the first coupler 20. In some embodiments, the joint may be configured to allow rotation within a single plane. For example, in some embodiments, the joint may be configured to rotate relative to the outer housing around a first axis (e.g., a central axis of a hinge pin 243 or similar), the first axis being orthogonal to a path 249 through the positioning device.

The path 249 may represents a path of a medical device from the proximal opening 260 through a lumen extending through the second coupler, through a lumen in a proximal end 248 of the flexible joint, through the second volume of space 268 through the first volume of space 244 and through to the distal end 262 of the outer housing, and through the first coupler 20. After passing the distal end of the outer housing, a medical device would then enter anti-contamination measures 270 of the introducer hub before entering a third volume of space 275 defined by the hub. The anti-contamination measures may include a hemostasis valve 271, a foam body 272, and/or cap 273.

Thus, the second volume of space may be configured to allow a medical device to be slidably received and directed from the proximal end 266 of the joint to the distal end 262 of the outer housing.

In some embodiments, the second volume of space 278 within ball 241 is configured to have two forks 245, 246. A first fork 245 may be utilized when the device is configured such that the path 249 at the proximal end 266 of the joint is substantially parallel to the path as it passes the distal end 262 of the outer housing. That is—when the joint is generally in a “straight” position, the first fork may be used. Conversely, the second form 246 may be utilized when the device is configured such that the path 249 at the proximal end 266 of the joint is at an angle relative to the path as it passes the distal end 262 of the outer housing. In this regard, the medical device (and or another accessory) may be passed through the hinge at a desired angle. In some embodiments, the angle may be between 30-90 degrees.

The flexible hinge may be positioned between the first coupler 20 and the second coupler 220, where the flexible hinge may be configured to allow the medical device to be slidably received through the flexible hinge.

The hinged section may be configured to allow the adjustable lock portion to rotate around the hinged section relative to the first coupler.

The positioning device may be configured such that a medical device can be slidably received through the adjustable locking portion, and through the first coupler.

FIG. 3C illustrates a perspective view of the positioning device of FIG. 3B, with the insertion of a medical device and catheter into the positioning device. As shown in this view, the positioning device has been adjusted such that the desired positioning of the medical device, repositioning sheath, or both, relative to an introducer hub has been achieved by the clinician. As shown in this view, the first coupler 20 is configured to be removably coupled to a proximal end of an introducer hub 81. The first coupler also may be configured to slidably receive a medical device (which may include catheter 280) and allow the medical device to enter into the introducer hub 81. After entering the introducer hub, the medical device may then enter into an introducer sheath 82, and then through an access point 291 on the surface of a patient's skin 290 and into the patient.

In some embodiments, the positioning device may include a lock 250 that may include an adjustable button 251 (e.g., a press button) coupled to the second coupler 30. In some embodiments, the lock may be configured to be allow or prevent a medical device and/or repositioning sheath from being adjusted axially relative to the second coupler 30 and to the hub based on the position of the medical device and/or repositioning sheath relative to the second coupler.

The positioning device may be configured such that a medical device (which may include catheter 280) can be slidably received through the lock 250, and through the first coupler 20 when the adjustable locking portion is in an unlocked configuration. When the adjustable locking portion is in a locked configuration, the medical device and/or repositioning sheath cannot move axially relative to the adjustable locking portion.

The positioning device may include a hinge configured to allow the lock to rotate around the hinge relative to the first coupler.

The positioning device may include a proximal end 222 configured to slidably receive the medical device (or a repositioning sheath).

The medical device may include one or more markings 285 on an external surface of a catheter 280. In some embodiments, the markings may be radiopaque. In some embodiments, the markings are visible markings. In some embodiments, at least one marking is located proximal to the positioning device.

In some embodiments, the positioning device is may include a fitting 295. The fitting may be configured to hold a transparent, flexible, sterile member 296 (e.g., a bag, etc.) that is positioned around at least part of the catheter 280. The fitting 295 may be coupled to the proximal end 222 through the transparent, flexible, sterile member 296.

In some embodiments, a kit may be provided, the kit may include an introducer hub, a repositioning sheath, and a positioning device as disclosed herein.

In some embodiments, a kit may be provided. The kit may include a medical device, a positioning device as disclosed herein, and optionally a repositioning sheath.

Referring to FIG. 3D, another embodiment of a housing 231 is illustrated. As shown in this view, the housing may include a two-piece socket with first and second housing members 233a, 233b that are attached together to form a housing and ring (similar to the housing and first coupler shown in FIG. 1J, for example). As will be appreciated in view of FIG. 3D, the clinician may rotate the second housing portion 233b around a hub flange (see the arrow in FIG. 3D) to lock and close the housing-hub interface. A closed housing-hub interface can be seen in FIG. 3E.

Referring to FIG. 3F, another multi-piece housing is illustrated. As shown in this view, after aligning the housing with the hub via corresponding alignment tabs, the hub can be locked relative to the housing by axially sliding clips 237 of the housing over the hub, e.g., over the hub flange (see the arrow in FIG. 3F). As will be appreciated, the shape and size of the clips may correspond to that of the hub (e.g., the hub flange). As will be further appreciated, although shown as having two clips, the housing may be configured with a single clip or more than two clips.

FIG. 3G shows still another way of attaching a housing 231 to a hub. As shown in this view, the housing may include first and second tabs 238a, 238b that can engage with the hub. In such embodiments, the tabs may be splayed outwardly and away from the hub while sliding over the hub flange, after which point the tabs may snap back into place and hold the hub to the housing. To release, the user can squeeze the tabs to again splay the tabs outwardly to allow the clinician to disengage the hub from the housing.

FIGS. 3H and 3I show yet another way of attaching the housing 231 to the hub 81. As shown in this view, the housing may include a hinged clamp that may be snapped over the hub (e.g., the hub flange). As with the other embodiments, the clamp may be attached after the housing is first aligned with the hub.

As will be appreciated, other methods of attaching the housing to the hub may be used. For example, a user may push forward (e.g., towards the hub) to lock and unlock as a protrusion (e.g., on the hub) slides into a ring on the housing or as a protrusion on the housing slides into a ring on the hub. In still another embodiment, a user may rotate a ring of the housing until it engages with the hub. Then, as the ring and hub engages and locks so does the ring and the housing.

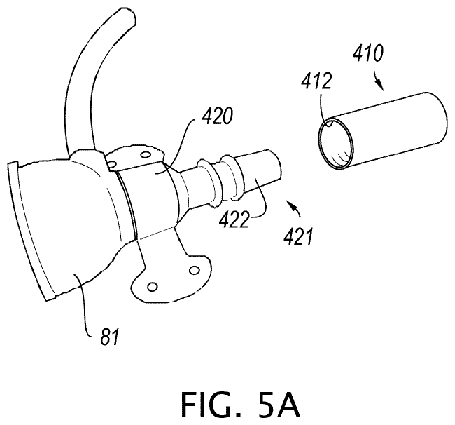

Referring to FIG. 4, in some embodiments, a method for adjusting positioning of a medical device, repositioning sheath, or both relative to an introducer hub may be provided. In embodiments having a lock, the method 300 may include unlocking 310 a lock on the positioning device. In some embodiments, the method may include adjusting 320 the positioning of a medical device, repositioning sheath, or both. The adjusting step may include: (1) rotating 321 a second coupler of the positioning device relative to the first coupler of the positioning device; (2) moving 322 the medical device, repositioning sheath, or both in an axial direction; or (3) a combination thereof.

The method may include locking 330 the locking mechanism on the positioning device. In some embodiments, locking the locking mechanism on the positioning device may include locking a locking mechanism to prevent the medical device, repositioning sheath, or both from moving axially relative to the locking mechanism. In some embodiments, the locking the locking mechanism may include locking a locking mechanism to prevent the proximal end of the positioning device from rotating relative to the distal end of the positioning device. In some embodiments, at least two locking mechanisms are adjusted, one to prevent axial movement, and one to prevent rotational movement.

The disclosed systems can be used in various applications. For example, currently, for certain conventional non-percutaneous (surgical) insertions, a practitioner will typically expose a blood vessel (e.g., the axillary artery) and attach a graft, then insert a peel-away sheath into the graft and secure the graft to the peel-away sheath. The peel-away sheath provides support for a medical device (such as a catheter-based pump), to be passed through the graft. The sheath also includes a hemostasis valve. At the end of the procedure, the graft is clamped, the sheath is removed and peeled apart to allow removal of the sheath while leaving the catheter in place in the graft, and then a repositioning unit including a butterfly portion that is preinstalled on a proximal portion of the catheter is slid into and attached to the graft before unclamping, thereby allowing the medical device to remain inserted through the blood vessel via the graft.

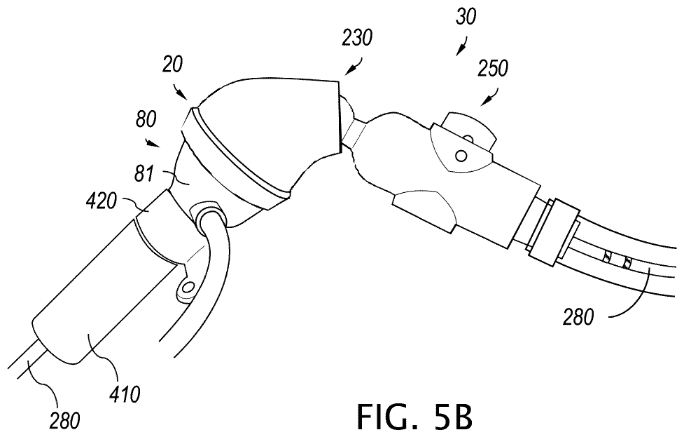

The disclosed system can eliminate the use of the peel-away sheath in the above-described procedure, thereby simplifying the insertion procedure for non-percutaneous applications. Referring to FIG. 5A, a graft 410 can be seen, with an inner surface 412 defining a lumen extending through the graft. A suture hub 420 (e.g., a butterfly) is seen, coupled to a hub body 81. The suture hub may be rotatably coupled to the hub body. The distal end 421 of the suture hub has an outer surface 422 that is configured to be removably coupled to the inner surface 412 of the graft.

Referring to the conventional procedure, after attaching the graft to the vessel and clamping the graft, using the disclosed systems, the distal end of the suture hub can be directly attached the graft. Then, the graft can be unclamped since the hub body includes a hemostasis valve.

A medical device can then be passed through. Referring to FIG. 5B, it can be seen that once the graft and repositioning butterfly are appropriately connected, a medical device (such as a medical device coupled to catheter 280, such as a catheter-based heart pump) can be passed through second coupler 30, the hinge 230, first coupler 20, hub body 81, the suture hub 420 and graft 410. In this manner, the hub and suture hub may provide sufficient support for the medical device through the graft.

It will be understood that internal dimensions of the hub (including the hub body 81) and the suture hub 420 may be appropriately selected based on, e.g., outer diameters of the medical device intended to be inserted through the hub and suture hub.

Embodiments of the present disclosure are described in detail with reference to the figures wherein like reference numerals identify similar or identical elements. It is to be understood that the disclosed embodiments are merely examples of the disclosure, which may be embodied in various forms. Well known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art.

Those skilled in the art will recognize or be able to ascertain using no more than routine experimentation many equivalents to the specific embodiments of the invention described herein. Such equivalents are intended to be encompassed by the following claims.

What is claimed is:

1. A positioning device comprising:
a first coupler configured to be removably coupled to a proximal end of an introducer hub; and
a second coupler configured to be removably coupled to a portion of a repositioning sheath or catheter that is proximal to the first coupler, the first coupler being attached to the second coupler;
wherein the positioning device is configured to maintain an axial position of repositioning sheath or catheter relative to the introducer hub.

2. The positioning device according to claim 1, further comprising an extension bridge extending between the first coupler and the second coupler, the extension bridge configured to maintain a fixed axial distance between the introducer hub and a proximal end of the repositioning sheath or catheter.

3-9. (canceled)

10. The positioning device according to claim 1, wherein the first coupler comprises a first surface containing a threaded portion configured to interact with one or more threads on an exterior surface of the introducer hub to prevent the first coupler from moving axially relative to the introducer hub.

11. The positioning device according to claim 1, wherein the first coupler includes at least one fastener configured to interact with a corresponding fastener on an exterior surface of the introducer hub to maintain a position of the introducer hub relative to the first coupler.

12. (canceled)

13. The positioning device according to claim 1, wherein the first coupler includes a rotatable portion and a non-rotatable portion, the rotatable portion located distal from the non-rotatable portion, the rotatable portion configured to interact with threads, tabs or extruded portions of an exterior surface of the introducer hub to prevent the first coupler from moving axially relative to the introducer hub.

14-17. (canceled)

18. The positioning device according to claim 1, further comprising an adjustable arm coupled to the first coupler, the adjustable arm configured to support the repositioning sheath or catheter as the repositioning sheath or catheter extends proximally from the first coupler.

19-21. (canceled)

22. The positioning device according to claim 1, wherein the positioning device comprises a hinge that hingedly connects a first section of the positioning device to a second section of the positioning device, wherein at least a portion of the introducer hub and at least a portion of the repositioning sheath or catheter can be placed at least partially into the first section before the second section hingedly closes.

23. (canceled)

24. The positioning device according to claim 22, further comprising a locking mechanism configured to prevent the positioning device from hingedly opening.

25-26. (canceled)

27. The positioning device according to claim 22, wherein an inner surface of the first section and/or second section comprises an indicator representing a correct positioning of the repositioning sheath.

28. The positioning device according to claim 22, wherein each of the first and second sections includes a length having one or more ribs extending into a channel formed between the first and second sections, the channel configured to receive at least a portion of the repositioning sheath and/or catheter.

29. The positioning device according to claim 1, wherein the first coupler includes a clamshell, and the second coupler is attached to the clamshell via a ball joint that is secured within the clamshell.

30. (canceled)

31. A positioning device comprising:
a first coupler having a housing and a means for locking the first coupler to an introducer sheath; and
a second coupler connected to the first coupler, the second coupler configured to receive a tubular member.

32. The positioning device according to claim 31, wherein the first coupler includes an alignment tab.

33. The positioning device according to claim 31, wherein the first coupler includes one or more indicia of a locking status of the first coupler.

34. The positioning device according to claim 31, wherein the first coupler is pivotably attached to the second coupler.

35. (canceled)

36. A positioning device configured for adjustable positioning of a medical device, repositioning sheath, catheter, or combination thereof, relative to an introducer hub, comprising:
a first coupler configured to be removably coupled to a proximal end of an introducer hub;
a second coupler configured to slidably receive the medical device, repositioning sheath, catheter, or combination thereof; and
a hinged section configured to allow an adjustable locking portion to rotate around the hinged section relative to the first coupler.

37. The positioning device according to claim 36, wherein the second coupler is configured to allow or prevent the medical device and/or repositioning sheath from being adjusted axially relative to the introducer hub based on a position of an adjustable lock, where the positioning device is configured such that a medical device can be slidably received through the adjustable locking portion, and through the first coupler.

38. The positioning device according to claim 36, wherein the hinged section can be unlocked to allow the hinged section to rotate, or locked to prevent the hinged section from rotating.

39. A positioning device configured for adjustable positioning of a medical device, repositioning sheath, or both, relative to an introducer hub, comprising:
a first coupler configured to be removably coupled to a proximal end of an introducer hub, the first coupler configured to slidably receive a medical device and allow the medical device to enter into the introducer hub;
a second coupler configured to slidably receive a portion of a repositioning sheath and/or catheter device; and
a flexible hinge connecting the first and second couplers, the flexible hinge configured to allow the medical device to be slidably received through the flexible hinge.

40. The positioning device according to claim 39, wherein the flexible hinge includes a lock configured to prevent the flexible hinge from rotating, wherein in an unlocked configuration the flexible hinge can rotate.

41. The positioning device according to claim 39, wherein the flexible hinge comprises:
an outer housing having a distal end, a proximal end, and an inner surface defining a first volume of space extending therethrough; and
a joint having a distal end, a proximal end, and an inner surface defining a second volume of space therethrough, the distal end of the joint positioned at least partially within the first volume of space, the joint being configured to rotate relative to the outer housing around a first axis, the first axis being orthogonal to a path from the distal end of the outer housing to the proximal end of the outer housing;
wherein the second volume of space is configured to allow a medical device to be slidably received and directed from the proximal end of the joint to the distal end of the outer housing.

42-52. (canceled)
